(19) United States
(12) Patent Application Publication
(10) Pub. No.: US 2026/0191888 A1
(43) Pub. Date: Jul. 9, 2026

(54) METHODS, COMPOSITIONS, AND KITS FOR TREATING CANCERS

(71) Applicants: The University of Hong Kong, Hong Kong (HK); Laboratory for Synthetic Chemistry and Chemical Biology Limited, Hong Kong (HK)

(72) Inventors: Alice Sze Tsai WONG, Hong Kong (HK); Ayon Ahmed HASSAN, Hong Kong (HK)

(21) Appl. No.: 19/382,301

(22) Filed: Nov. 7, 2025

Related U.S. Application Data
(60) Provisional application No. 63/717,831, filed on Nov. 7, 2024.

(51) Int. Cl. A61K 31/7008; A61K 39/00; A61K 39/395; A61P 35/00; C07K 16/28

(52) U.S. Cl. CPC A61K 31/7008; A61K 39/3955; A61P 35/00; C07K 16/2863; A61K 2039/505

(57) ABSTRACT

A method for treating cancer by targeting aberrant sialylation of the insulin-like growth factor 1 receptor (IGF1R) to enhance therapeutic responsiveness is provided. The method includes administering to a subject in need thereof a pharmaceutically effective amount of a sialyltransferase inhibitor (STI) and a pharmaceutically effective amount of an IGF1R-targeting agent. The STI inhibits the expression or enzymatic activity of ST6GAL1, thereby reducing α2,6-linked sialylation of the asparagine 607 residue of IGF1R. This desialylation enhances the accessibility of IGF1R to the IGF1R-targeting agent, improving its binding affinity and inhibitory efficacy against IGF1R activation and downstream oncogenic signaling.

### CROSS-REFERENCE TO RELATED APPLICATIONS

The present application claims priority from U.S. Provisional Utility Patent application no. 63/717,831 filed Nov. 7, 2024; the disclosure of which is incorporated herein by reference in its entirety.

### REFERENCE TO SEQUENCE DISCLOSURE

The sequence listing file under the file name “P3377US01_Sequence Listing.xml” submitted in ST.26 XML file format with a file size of 7,235 bytes created on Nov. 6, 2025 and filed on Nov. 7, 2025 is incorporated herein by reference.

### FIELD OF THE INVENTION

The present invention generally relates to the field of biopharmaceuticals and oncology therapeutics. More specifically the present invention relates to methods, compositions, and kits for enhancing the efficacy of insulin-like growth factor 1 receptor (IGF1R)-targeted therapies through modulation of tumor glycosylation.

### BACKGROUND OF THE INVENTION

Receptor tyrosine kinases (RTKs) are frequently hyperactivated in human malignancies and represent key therapeutic targets for cancer treatment. Multiple FDA-approved therapeutics currently target RTKs, underscoring their clinical relevance. Among these, the insulin-like growth factor 1 receptor (IGF1R) plays a central role in regulating cellular proliferation, survival, and metastatic potential across diverse cancer types. Over the past two decades, IGF1R has emerged as one of the most extensively studied molecular targets in oncology. Dysregulated IGF1R signaling has been documented in at least fifteen different cancers, suggesting that effective modulation of this pathway could yield broad therapeutic benefits. Despite the development of more than twenty-five IGF1R inhibitors and monoclonal antibodies that have entered clinical trials, including ganitumab, the overall clinical outcomes have been disappointing, particularly in ovarian cancer, where no IGF1R-targeting therapy has successfully advanced beyond Phase II trials. This limited efficacy highlights a critical need to understand and overcome the mechanisms underlying resistance to IGF1R-targeting agents such as ganitumab.

Ovarian cancer remains one of the most lethal gynecological malignancies, characterized by late diagnosis, high recurrence rates, and resistance to standard chemotherapeutic regimens. Although targeted therapies and immunomodulatory agents have expanded the therapeutic landscape, they have not significantly improved long-term survival outcomes. The intrinsic resistance to IGF1R inhibitors and monoclonal antibodies, including ganitumab, further underscores the complexity of ovarian cancer signaling networks and the need for novel combinatorial or sensitization strategies. Accordingly, there is an urgent demand for therapeutic approaches that can enhance the responsiveness of ovarian cancer cells to IGF1R-targeting agents, restore drug sensitivity, and achieve more durable disease control.

The present invention addresses this need by identifying molecular mechanisms and therapeutic interventions that improve the response to IGF1R-targeting therapies such as ganitumab. Through modulation of key signaling pathways and cellular components associated with IGF1R activity, the invention provides a strategy for overcoming therapeutic resistance, thereby enhancing the efficacy of IGF1R inhibition in ovarian cancer and other malignancies driven by aberrant IGF1R signaling.

### SUMMARY OF THE INVENTION

It is an objective of the present invention to provide methods, compositions, and kits to solve the aforementioned technical problems.

In accordance with a first aspect of the present invention, a method of treating a cancer in a subject in need thereof is provided. Specifically, the method includes the following steps: administering a pharmaceutically effective amount of a sialyltransferase inhibitor (STI) to the subject; and administering a pharmaceutically effective amount of an insulin-like growth factor 1 receptor (IGF1R)-targeting agent to the subject.

The STI inhibits the expression or enzymatic activity of ST6GAL1, thereby reducing α2,6-linked sialylation of the asparagine 607 residue of IGF1R, and enhancing the binding and inhibitory activity of the IGF1R-targeting agent.

In accordance with one embodiment, the STI includes 3Fax-Peracetyl Neu5Ac or a pharmaceutically acceptable salt or derivative thereof or a small-molecule inhibitor of ST6GAL1.

In accordance with another embodiment, the IGF1R-targeting agent includes Ganitumab.

In accordance with yet another embodiment, the IGF1R-targeting agent is an anti-IGF1R antibody, an IGF1R ligand-binding inhibitor, or a small molecule IGF1R tyrosine kinase inhibitor.

In accordance with yet another embodiment, the cancer includes breast cancer, lung cancer, colorectal cancer, prostate cancer, pancreatic cancer, liver cancer, esophageal cancer, gastric cancer, ovarian cancer, cervical cancer, endometrial cancer, renal cancer, bladder cancer, thyroid cancer, melanoma, head and neck cancer, or sarcoma.

In accordance with yet another embodiment, the cancer is ovarian cancer.

In accordance with yet another embodiment, the subject exhibits elevated ST6GAL1 expression in the tumor tissue prior to treatment.

In accordance with yet another embodiment, the method further includes identifying a subject having a tumor with high ST6GAL1 expression or increased α2,6-sialylation of IGF1R, and selecting the subject for co-administration of the STI and IGF1R-targeting agent.

In accordance with yet another embodiment, the STI and the IGF1R-targeting agent are co-administered simultaneously, sequentially, or separately.

In accordance with yet another embodiment, the combined administration of the STI and the IGF1R-targeting agent produces a synergistic reduction in tumor growth relative to either treatment alone.

In accordance with a second aspect of the present invention, a pharmaceutical composition for treating cancer is provided. The pharmaceutical composition includes: a pharmaceutically effective amount of a STI; a pharmaceutically effective amount of an IGF1R-targeting agent; and a pharmaceutically acceptable carrier or excipient.

In accordance with one embodiment, the STI includes 3Fax-Peracetyl Neu5Ac or a pharmaceutically acceptable salt or derivative thereof or a small-molecule inhibitor of ST6GAL1.

In accordance with another embodiment, the IGF1R-targeting agent includes Ganitumab.

In accordance with yet another embodiment, the IGF1R-targeting agent is an anti-IGF1R antibody, an IGF1R ligand-binding inhibitor, or a small molecule IGF1R tyrosine kinase inhibitor.

In accordance with a third aspect of the present invention, a kit of enhancing the therapeutic effect of an IGF1R-targeting agent is provided. Specifically, the kit includes the following components: a pharmaceutically effective amount of a STI; and a pharmaceutically effective amount of an IGF1R-targeting agent. The STI inhibits the expression or activity of ST6GAL1, thereby preventing sialylation of IGF1R and improving binding of the IGF1R-targeting agent to IGF1R.

In accordance with one embodiment, the STI includes 3Fax-Peracetyl Neu5Ac or a pharmaceutically acceptable salt or derivative thereof or a small-molecule inhibitor of ST6GAL1.

In accordance with another embodiment, the IGF1R-targeting agent includes Ganitumab.

In accordance with yet another embodiment, the IGF1R-targeting agent is an anti-IGF1R antibody, an IGF1R ligand-binding inhibitor, or a small molecule IGF1R tyrosine kinase inhibitor.

### BRIEF DESCRIPTION OF THE DRAWINGS

Embodiments of the invention are described in more details hereinafter with reference to the drawings, in which:

FIGS. 1A-1B depict that the desialylation sensitizes ovarian cancer cells to anti-IGF1R mAb Ganitumab, in which: FIG. 1A depicts the colony formation assay of ovarian cancer cells treated with Ganitumab only or in combination with fucosyltransferase inhibitor (FTI)/sialyltransferase inhibitor (STI); and FIG. 1B depicts the immunoprecipitation of sialidase- or fucosidase-treated IGF1R with Ganitumab;

FIGS. 2A-2C depict that desialylation sensitizes ovarian cancer cells to AG1024 and Ganitumab, in which: FIG. 2A depicts the MTT assay of ovarian cancer cells treated with AG1024 only or in combination with FTI/STI; and FIG. 2B and FIG. 2C respectively depict the SNA (FIG. 2B) and (FIG. 2C) Ganitumab staining of formalin fixed paraffin embedded section from ovarian cancer cell block treated with STI;

FIGS. 3A-3G depict that IGF1R is modified with α2-6-linked sialylated N-glycans on amino acids 607 and 913, in which: FIG. 3A depicts the western blot of sialidase-treated IGF1R; FIG. 3B shows the SNA lectin blot of sialidase-treated, immunoprecipitated IGF1R; FIG. 3C depicts the western Blot of IGF1R with combination of STI and PNGaseF treatment; FIG. 3D displays the western blot of IGF1R after PNGaseF treatment and SNA pulldown; FIG. 3E shows the western blot of IGF1R after STI treatment and SNA pulldown; FIG. 3F depicts the western blot of IGF1R after SNA pull down in breast, lung, and colon cancer cells; and FIG. 3G shows the annotation of identified sialylated glycan;

FIGS. 4A-4E depict that the asparagine 607 sialylation of IGF1R inhibits binding and response to Ganitumab, in which: FIG. 4A depicts the western blot of IGF1R alpha and beta subunits in wildtype and N607Q mutant; FIG. 4B shows the western blot of IGF1R N607Q after SNA pulldown; FIG. 4C depicts the western Blot of IGF1R N607Q immunoprecipitated with Ganitumab; FIG. 4D displays the western blot of p-IGF1R N607Q after treatment with Ganitumab; and FIG. 4E depicts the colony formation of IGF1R N607Q treated with Ganitumab;

FIGS. 5A-5D depict that the mutating asparagine 607 to glutamine sensitizes IGF1R to Ganitumab in an in vivo xenograft model of ovarian cancer, in which: FIG. 5A and FIG. 5B respectively depict the bioluminescence imaging (FIG. 5A) and mesenteric tumor nodules (FIG. 5B) of Ganitumab-treated ovarian cancer cells harboring N607Q IGF1R mutation; FIG. 5C depicts the H&E staining of tumors harvested from FIG. 5A and FIG. 5B; and FIG. 5D shows the IHC stain of p-IGF1R of tumors harvested from FIG. 5A and FIG. 5B;

FIGS. 6A-6B depict that ST6GAL2 does not sialylate IGF1R, in which: FIG. 6A shows the indel spectrum of ST6GAL2 sgRNA-transduced cells assessed by Tracking of Indels by DEcomposition (TIDE); and FIG. 6B depicts the western blot of IGF1R from ST6GAL2 sgRNA-transduced cells immunoprecipitated with Ganitumab;

FIG. 7 is a schematic of desialylation-mediated sensitization of ovarian cancer cells to Ganitumab;

FIGS. 8A-8G depict that inhibition of sialylation using STI prevents the addition of sialic acids to IGF1R and significantly enhances the therapeutic response to Ganitumab, in which: FIG. 8A and FIG. 8B respectively show SNA staining and SNA pulldown results demonstrating that STI treatment successfully inhibits sialylation of IGF1R; FIG. 8C shows immunohistochemical staining indicating that Ganitumab exhibits increased binding affinity to STI-treated ovarian cancer cells but not to normal ovarian surface epithelial (OSE) cells; FIG. 8D depicts that STI treatment significantly improves Ganitumab efficacy, as evidenced by a marked reduction in cell survival in the co-treated group compared to Ganitumab alone; FIG. 8E and FIG. 8F depict that in vivo co-treatment with STI enhances the therapeutic efficacy of Ganitumab, as demonstrated by bioluminescence imaging (FIG. 8E) and mesenteric tumor nodule quantification (FIG. 8F); and FIG. 8G shows the SNA lectin staining of tumor tissues harvested from FIG. 8E and FIG. 8F;

FIGS. 9A-9C depict that ST6GAL1 is upregulated in multiple cancers and is responsible for sialylation of IGF1R, in which: FIG. 9A depicts SNA pulldown analysis results; FIG. 9B depicts the IP assay results; and FIG. 9C depicts the Western blot analysis results; and

FIGS. 10A-10C depict that increased expression of ST6GAL1 inhibits Ganitumab binding in patient tumor samples, in which: FIG. 10A shows that analysis of The Cancer Genome Atlas (TCGA) database reveals ST6GAL1 expression is significantly upregulated across multiple cancer types; FIG. 10B demonstrates that ST6GAL1 expression levels are inversely correlated with Ganitumab binding to ovarian cancer patient tissues; and FIG. 10C depicts that enzymatic removal of sialic acids enhances Ganitumab binding to ST6GAL1-high patient tumors.

### DETAILED DESCRIPTION

In the following description, methods, compositions, and/or kits of treating cancers and/or enhancing the therapeutic effect of a IGFbR-targeting agent and the likes are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

As used herein, the term “Ganitumab” is a fully humanized monoclonal antibody that specifically targets IGF1R, a receptor tyrosine kinase involved in cellular growth, differentiation, and survival signaling. Mechanistically, Ganitumab binds to the extracellular domain of IGF1R, blocking the binding of its natural ligands, IGF-1 and IGF-2, thereby preventing receptor activation, autophosphorylation, and downstream signaling through the PI3K/AKT and MAPK pathways. This results in reduced proliferation and increased apoptosis in tumor cells that are dependent on IGF1R signaling.

The present invention demonstrates that sialylation, and fucosylation of the IGF1R play a pivotal role in promoting peritoneal dissemination of ovarian cancer. Furthermore, the invention investigates how these glycoconjugates contribute to therapeutic resistance against IGF1R inhibition, with a particular focus on ganitumab—the only humanized monoclonal antibody against IGF1R that has progressed to clinical trials.

Glycosylation, the covalent attachment of glycan moieties to proteins and lipids, is increasingly recognized as a key regulatory mechanism in cancer initiation, progression, and metastasis. Recent studies have established that fucosylated and sialylated glycans can promote tumor growth, immune evasion, and metastatic spread. The present invention provides direct experimental evidence that α2,6-sialylation of IGF1R confers resistance to inhibition by ganitumab. Through systematic characterization of IGF1R-associated sialic acids, it is shown that these glycans are predominantly α2,6-linked and attached to N-glycans carried by asparagine residue 607. This specific modification is also detected in multiple cancer types where ganitumab has demonstrated limited clinical efficacy, including lung, breast, and colorectal cancers. Mutation of this asparagine residue to glutamine significantly reduces IGF1R sialylation, increases ganitumab binding affinity, and sensitizes tumors to IGF1R inhibition in xenograft models of ovarian cancer. Importantly, the invention further identifies ST6Gal I as the sialyltransferase responsible for catalyzing this modification and demonstrates that desialylation of ST6Gal I-high patient tumors enhances ganitumab binding. Collectively, these findings reveal that targeting IGF1R sialylation represents a promising therapeutic strategy for overcoming resistance to IGF1R inhibitors and monoclonal antibodies in otherwise refractory ovarian carcinomas.

IGF1R has long been recognized as an attractive therapeutic target; however, the underlying reason for the repeated clinical failures of IGF1R-targeting agents remains unclear. The present invention identifies a previously unrecognized post-translational modification of IGF1R that accounts for this resistance. Specifically, it has been discovered that α2,6-sialylation of IGF1R at asparagine residue 607, catalyzed by the sialyltransferase ST6Gal I, interferes with antibody-receptor binding. This modification sterically and electrostatically hinders the interaction between IGF1R and ganitumab, an anti-IGF1R monoclonal antibody, thereby diminishing its therapeutic efficacy. The present invention demonstrates that removal of sialic acid residues sensitizes ST6GAL1-high tumors to ganitumab, restoring drug binding and downstream inhibition of IGF1R signaling. Accordingly, the present invention provides a strategy for improving the responsiveness of ovarian cancer to ganitumab through targeted modulation of IGF1R sialylation. Moreover, since α2,6-sialylation of IGF1R is also observed in several other malignancies, this discovery has broad implications for overcoming resistance to IGF1R-targeted therapies in multiple cancer types.

Ovarian cancer remains a particularly challenging malignancy due to its asymptomatic progression and late-stage diagnosis, by which time most patients present with disseminated disease. Despite standard first-line treatment using cisplatin and paclitaxel, the five-year survival rate remains below 25%. There is an urgent need for novel molecular targets and sensitization strategies to improve this poor prognosis. Although ganitumab-mediated IGF1R inhibition has shown promise in preclinical models, its clinical efficacy in advanced ovarian cancer has been limited by intrinsic or acquired resistance. The present invention reveals that α2,6-sialylation of IGF1R is a principal mechanism underlying such resistance, and demonstrates that removal or inhibition of this modification restores ganitumab sensitivity, leading to improved therapeutic outcomes.

Sialylation has recently emerged as a critical regulator of RTK signaling and drug response across several cancers. For instance, in colorectal cancer, sialylation of the EGFR modulates its activation and sensitivity to monoclonal antibody cetuximab and the small-molecule inhibitor gefitinib. In gastric cancer, sialylation has been shown to affect responsiveness to crizotinib and trastuzumab by altering the sialylation status of RTKs such as MET, RON, and ErbB2. These studies collectively indicate that sialylated RTKs tend to be more resistant to targeted therapeutics. However, the glycosylation landscape is complex, and previous reports have generally failed to pinpoint the precise glycosylation site, the responsible enzyme, or the specific glycoform mediating therapeutic resistance. Without such detailed mechanistic understanding, translation of these findings into clinical applications has remained limited. The present invention addresses this knowledge gap by identifying the exact sialylation site (Asn607) on IGF1R and the specific sialyltransferase (ST6Gal I) responsible for the modification, thereby enabling targeted modulation of this resistance mechanism.

ST6Gal I is markedly upregulated in multiple solid tumors and represents the predominant enzyme responsible for the addition of α2,6-linked sialic acids to cell surface glycoproteins. Prior in vitro studies have implicated ST6Gal I in mediating drug resistance; however, patient-level evidence has been scarce. Duarte et al. reported the presence of α2,6-sialic acid residues on ErbB2 receptors in gastric cancer samples, yet the functional relationship between ST6GAL1 expression and antibody-receptor binding remained uncharacterized. In contrast, the present invention establishes a direct mechanistic link between ST6GAL1-mediated α2,6-sialylation of IGF1R and impaired ganitumab efficacy, demonstrating that targeted removal or inhibition of this modification restores therapeutic binding, inhibits IGF1R signaling, and enhances antitumor response.

In accordance with a first aspect of the present invention, a therapeutic method for treating cancers by targeting aberrant sialylation of the IGF1R is provided. The method involves administering to a subject in need thereof a pharmaceutically effective amount of a STI and a pharmaceutically effective amount of an IGF1R-targeting agent. The STI inhibits the expression or enzymatic activity of ST6GAL1, a key sialyltransferase responsible for catalyzing the addition of α2,6-linked sialic acids to N-glycan residues on IGF1R. By blocking this modification, the STI reduces α2,6-linked sialylation at the asparagine 607 residue of IGF1R, thereby enhancing the accessibility and binding affinity of the IGF1R-targeting agent to its receptor and improving its inhibitory activity against IGF1R signaling.

In one embodiment, the STI used in the present method comprises 3Fax-Peracetyl Neu5Ac or a pharmaceutically acceptable salt or derivative thereof or a small-molecule inhibitor of ST6GAL1. This compound functions as a broad-spectrum sialyltransferase inhibitor that competitively blocks sialic acid incorporation into glycoproteins, effectively desialylating IGF1R. The desialylated IGF1R exhibits increased sensitivity to inhibition by IGF1R-targeting agents, thereby potentiating anti-tumor efficacy.

In another embodiment, the IGF1R-targeting agent is Ganitumab, a humanized monoclonal antibody that binds to the extracellular domain of IGF1R, preventing ligand-induced receptor activation and downstream signaling through pathways such as PI3K/AKT and MAPK. Alternatively, the IGF1R-targeting agent may be any anti-IGF1R antibody, IGF1R ligand-binding inhibitor, or small molecule IGF1R tyrosine kinase inhibitor that blocks receptor activation. The present invention is applicable to a wide variety of solid tumors in which IGF1R signaling plays a key role, including but not limited to breast, lung, colorectal, prostate, pancreatic, liver, esophageal, gastric, ovarian, cervical, endometrial, renal, bladder, thyroid, melanoma, head and neck, and sarcoma cancers. In one preferred embodiment, the cancer is ovarian cancer, in which IGF1R hyperactivation and ST6GAL1 overexpression are often associated with resistance to IGF1R-targeted therapies.

The present invention further contemplates that the subject selected for treatment exhibits elevated ST6GAL1 expression in the tumor tissue prior to therapy. In this case, the method may include an initial step of identifying a patient whose tumor displays high ST6GAL1 expression or increased α2,6-sialylation of IGF1R. Such detection can be performed using immunohistochemistry, lectin staining, or gene expression assays. Based on this diagnostic information, the patient is selected for co-administration of the STI and the IGF1R-targeting agent, allowing precision medicine approaches that enhance the efficacy of anti-IGF1R therapies in resistant tumors.

The STI and IGF1R-targeting agent may be co-administered simultaneously, sequentially, or separately, depending on the desired pharmacokinetic profile and therapeutic strategy. Co-administration of these two agents produces a synergistic reduction in tumor growth compared to administration of either compound alone, as desialylation of IGF1R facilitates stronger and more sustained binding of the therapeutic antibody, resulting in enhanced receptor inhibition, decreased downstream signaling, and increased tumor cell apoptosis.

The present invention provides an innovative combination strategy that targets glycosylation-mediated resistance to IGF1R inhibition. By modulating the tumor glycome through inhibition of ST6GAL1-mediated α2,6-sialylation, the invention enables improved receptor targeting and significantly enhances the therapeutic response to IGF1R-directed treatments such as Ganitumab. This approach improves clinical outcomes across a broad spectrum of solid tumors characterized by ST6GAL1 upregulation and hypersialylation of IGF1R.

In accordance with a second aspect of the present invention, a pharmaceutical composition designed for the treatment of cancer by targeting aberrant sialylation of the IGF1R to enhance therapeutic responsiveness is provided. The pharmaceutical composition includes a pharmaceutically effective amount of a STI, a pharmaceutically effective amount of an IGF1R-targeting agent, and a pharmaceutically acceptable carrier or excipient suitable for clinical administration. The combination of these two active agents achieves a synergistic anti-tumor effect by simultaneously blocking the enzymatic addition of α2,6-linked sialic acids to IGF1R and enhancing the receptor's accessibility to targeted inhibition, thereby suppressing IGF1R signaling and downstream oncogenic pathways.

In one embodiment, the STI includes 3Fax-Peracetyl Neu5Ac or a pharmaceutically acceptable salt or derivative thereof or a small-molecule inhibitor of ST6GAL1. This compound acts as a potent inhibitor of sialyltransferase activity, particularly that of ST6GAL1, which catalyzes α2,6-sialylation of IGF1R at the asparagine 607 residue. By reducing or preventing this post-translational modification, the STI restores receptor conformation and increases the binding affinity of IGF1R-targeting agents. The inclusion of 3Fax-Peracetyl Neu5Ac in the composition therefore provides an effective means to desialylate tumor-associated IGF1R, sensitizing cancer cells to subsequent receptor blockade.

In another embodiment, the IGF1R-targeting agent comprises Ganitumab. The desialylation effect induced by the STI enhances Ganitumab's receptor-binding efficiency, resulting in increased therapeutic potency and duration of action.

Alternatively, the IGF1R-targeting agent may be selected from a group consisting of anti-IGF1R antibodies, IGF1R ligand-binding inhibitors, or small molecule IGF1R tyrosine kinase inhibitors. These compounds may act through direct receptor antagonism, competitive inhibition at the ligand-binding site, or suppression of kinase catalytic activity. When formulated together with the STI, these agents exhibit a cooperative mechanism of action in which the STI alleviates steric hindrance caused by sialylation, thereby improving the accessibility and effectiveness of the IGF1R-targeting inhibitor.

The pharmaceutical composition of the present invention can be formulated in various dosage forms, including but not limited to solutions, suspensions, emulsions, lyophilized powders, or sustained-release preparations. The composition may be administered via intravenous, subcutaneous, or intraperitoneal routes, depending on the therapeutic requirement. The pharmaceutically acceptable carrier or excipient may include buffers, stabilizers, solubilizers, surfactants, or preservatives that maintain the stability and bioavailability of both the STI and the IGF1R-targeting agent.

The present pharmaceutical composition provides a dual-targeted therapeutic approach that simultaneously modulates tumor glycosylation and receptor signaling. By incorporating both a sialyltransferase inhibitor such as 3Fax-Peracetyl Neu5Ac and an IGF1R-targeting agent such as Ganitumab, the invention offers a synergistic therapeutic platform that overcomes sialylation-mediated drug resistance and enhances clinical efficacy in the treatment of cancers characterized by elevated ST6GAL1 expression and hypersialylated IGF1R.

In accordance with a third aspect of the present invention, a kit designed to enhance the therapeutic efficacy of an IGF1R-targeting agent by modulating glycosylation-mediated drug resistance is further provided. The kit includes a pharmaceutically effective amount of a STI and a pharmaceutically effective amount of an IGF1R-targeting agent. The STI functions to inhibit the expression or enzymatic activity of ST6GAL1, thereby preventing α2,6-linked sialylation of IGF1R. This desialylation process restores receptor accessibility, enhances antibody-receptor affinity, and improves the inhibitory efficiency of the IGF1R-targeting agent against receptor activation and downstream signaling. As a result, the combined use of these two therapeutic components synergistically suppresses IGF1R-driven oncogenic pathways and improves treatment outcomes in cancer patients.

In one embodiment, the STI of the kit comprises 3Fax-Peracetyl Neu5Ac or a pharmaceutically acceptable salt or derivative thereof. This compound is a potent metabolic inhibitor of sialyltransferase activity and effectively prevents the incorporation of sialic acid into glycoproteins. Inhibition of ST6GAL1 by 3Fax-Peracetyl Neu5Ac reduces α2,6-sialylation of the asparagine 607 residue of IGF1R, thus exposing the receptor epitope that interacts with therapeutic antibodies such as Ganitumab. The kit may alternatively include other small-molecule inhibitors of ST6GAL1, such as FCW393 or transition-state analogs that selectively bind to the catalytic domain of the enzyme, thereby preventing the formation of sialylated glycan linkages. These inhibitors can be used individually or in combination, depending on the desired therapeutic regimen and pharmacokinetic properties.

In another embodiment, the IGF1R-targeting agent included in the kit comprises Ganitumab. Alternatively, the IGF1R-targeting agent may include other therapeutic classes such as anti-IGF1R antibodies, IGF1R ligand-binding inhibitors, or small-molecule tyrosine kinase inhibitors that block the intracellular kinase domain of IGF1R. These agents may be selected based on tumor type, receptor expression level, and prior responsiveness to IGF1R-targeted therapy.

The combination of an STI and an IGF1R-targeting agent in the kit provides a rational therapeutic strategy for overcoming glycosylation-mediated resistance in tumors exhibiting elevated ST6GAL1 expression. The kit may be configured for co-administration or sequential administration, allowing clinicians to tailor dosing schedules to optimize desialylation prior to receptor inhibition. The components may be formulated separately or in a unified pharmaceutical preparation, with each unit comprising a pharmaceutically acceptable carrier or excipient suitable for clinical use. The carriers may include saline, phosphate-buffered saline, stabilizers, surfactants, or preservatives that maintain the stability and bioactivity of both the STI and the antibody.

The kit may also include instructions for clinical administration, dosage recommendations, and diagnostic guidance for selecting patients likely to benefit from the combination therapy. In particular, patients whose tumors exhibit high ST6GAL1 expression or increased α2,6-sialylation of IGF1R may be identified through immunohistochemistry, lectin staining, or molecular assays, and subsequently selected for treatment with the disclosed kit. By providing both desialylation and receptor inhibition components, the present invention offers an integrated therapeutic solution that enhances the efficacy of IGF1R-targeted therapies such as Ganitumab, overcomes resistance mechanisms in hypersialylated tumors, and improves clinical outcomes across a wide range of cancer types.

### EXAMPLES

### Example 1. Desialylation Sensitizes Ovarian Cancer Cells to Anti-IGF1R mAb Ganitumab

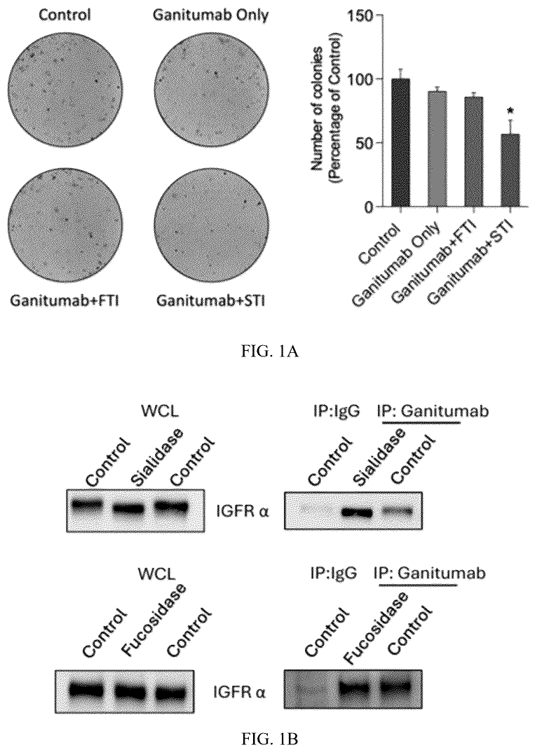

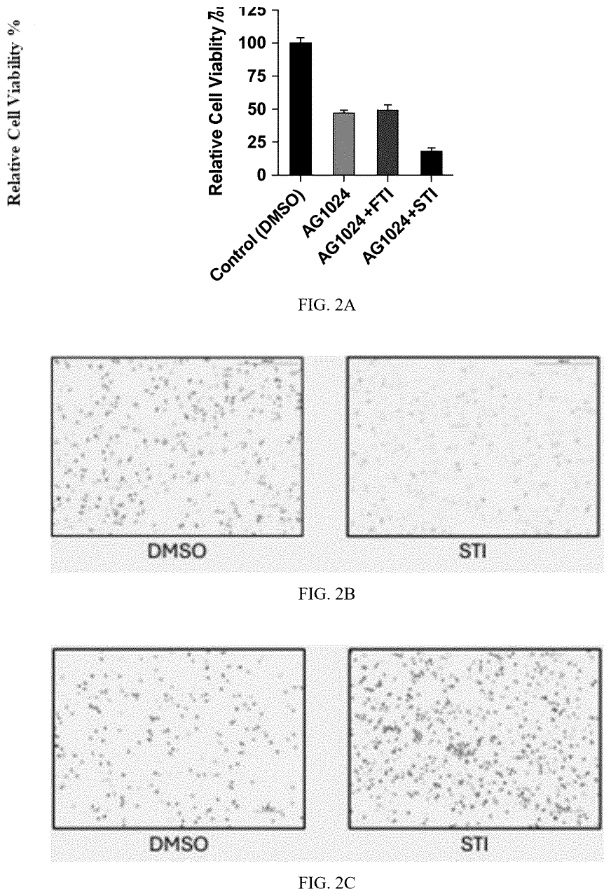

To determine whether sialylation or fucosylation modulates the sensitivity of ovarian cancer cells to ganitumab, colony formation assays are performed in the presence of either a fucosyltransferase inhibitor (FTI) or a STI, in combination with ganitumab (FIG. 1A). The results demonstrate that treatment with STI, but not FTI, markedly reduces the colony-forming ability of ovarian cancer cells under ganitumab exposure. This finding indicates that desialylation sensitizes ovarian cancer cells to ganitumab. Similar results are obtained when FTI or STI is combined with the small-molecule IGF1R inhibitor AG1024, as assessed by MTT assay (FIG. 2A). To further evaluate whether sialylation or fucosylation directly affects the binding interaction between ganitumab and IGF1R, immunoprecipitation (IP) assays are performed using IGF1R treated with sialidase or fucosidase, followed by incubation with ganitumab (FIG. 1). Desialylation significantly increases the amount of IGF1R recovered in the IP eluate, indicating enhanced physical binding of desialylated IGF1R to ganitumab. Consistent with these results, immunohistochemical (IHC) staining of STI-treated ovarian cancer cell blots using ganitumab reveals a marked increase in signal intensity (FIG. 2B), confirming that desialylation enhances ganitumab binding at the cellular level.

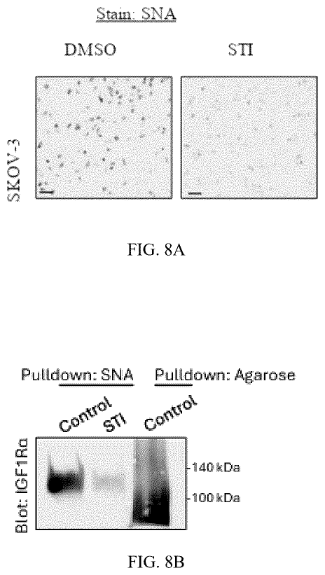

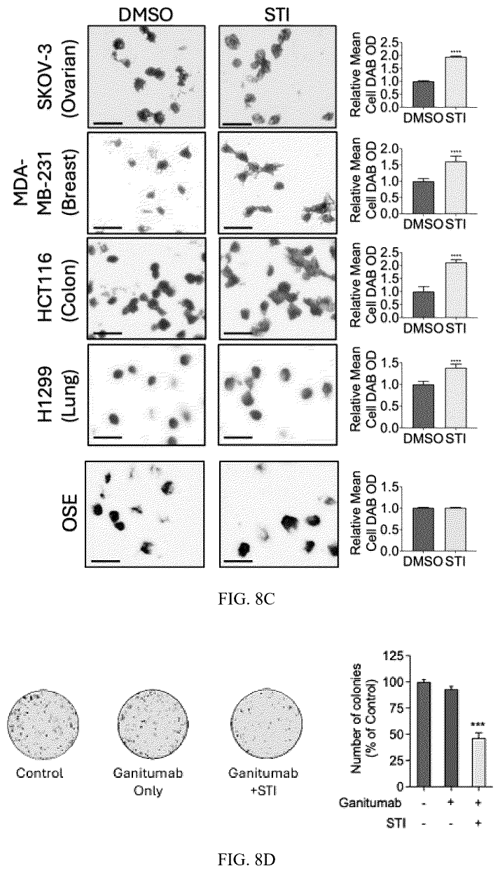

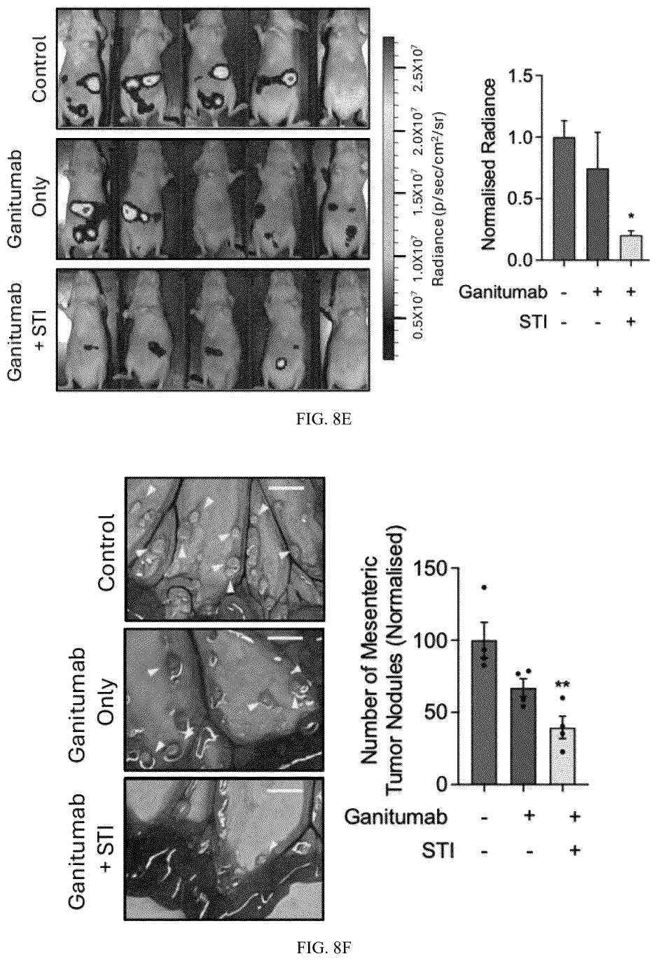

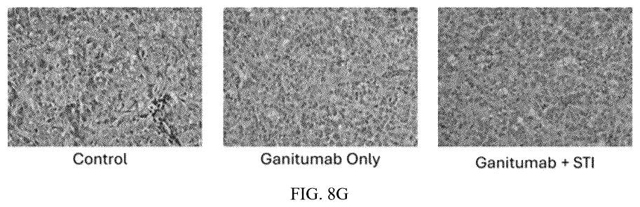

Building upon the observed enhancement of ganitumab-IGF1R interaction following desialylation, the effect of this modification on therapeutic efficacy is examined in a xenograft model of ovarian cancer metastasis. Ovarian cancer cells are injected intraperitoneally into mice and treated with ganitumab alone or in combination with STI. Bioluminescence imaging reveals significantly fewer residual tumor cells in mice receiving the combined STI and ganitumab treatment compared to those treated with ganitumab alone (FIG. 8E). Lectin histochemical analysis confirms that STI administration effectively prevents sialylation within tumor tissues (FIG. 8G). Consistent with these findings, macroscopic examination of the mesenteric membrane demonstrates a substantial reduction in the number of visible tumor nodules in STI-treated mice (FIG. 8F). Collectively, these results demonstrate that inhibition of sialylation enhances the antitumor activity of ganitumab, both by promoting IGF1R binding and by suppressing metastatic tumor burden in vivo.

### Example 2. IGF1R is Modified with α2,6-Linked Sialylated N-Glycans on Asparagine Residues 607 and 913

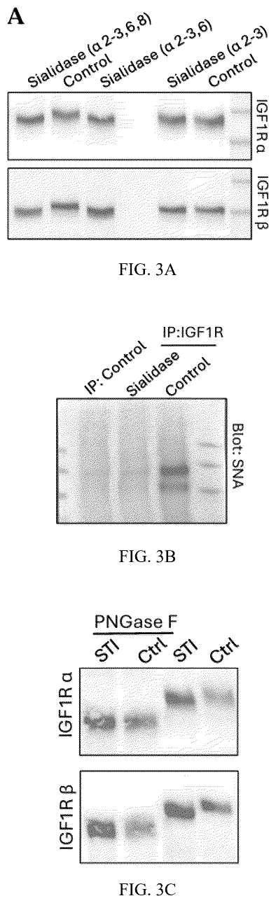

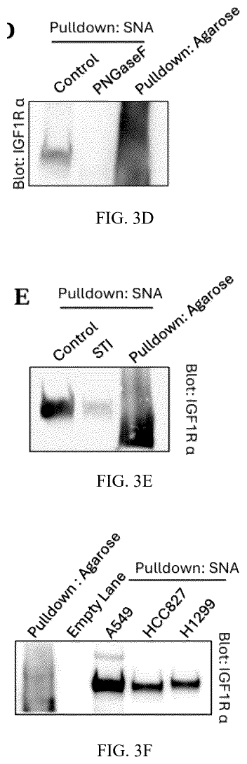

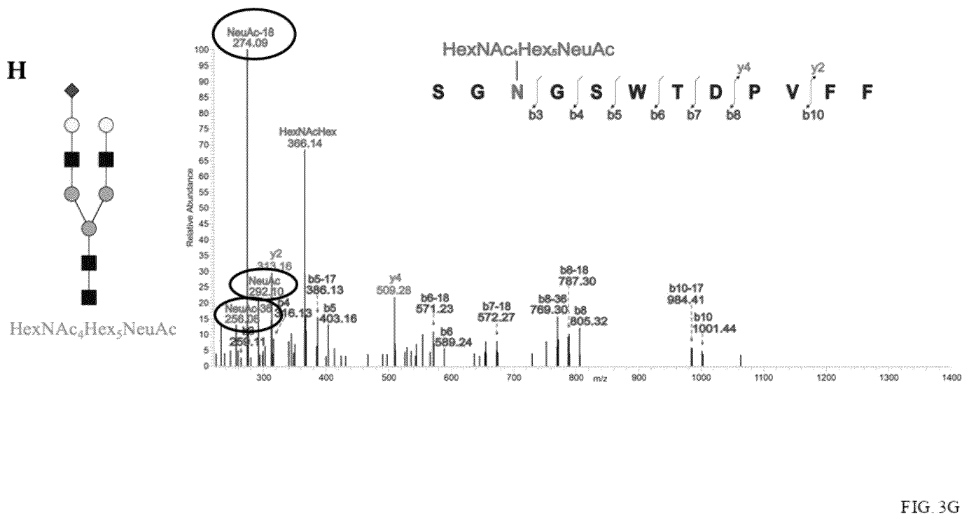

To characterize the sialic acid modifications present on IGF1R, a combination of lectin binding assays, glycosidase digestions, and mass spectrometric analyses is employed. In humans, sialic acids can occur in α2-3, α2-6, or α2-8 linkages and may be attached to either N- or O-glycans. Whole-cell lysates are digested with a panel of sialidases that selectively cleave α2-3, α2-3,6, or α2-3,6,8-linked sialic acids. Based on the observed band-shift patterns of the IGF1R α- and β-subunits, it is determined that the majority of sialic acids on IGF1R are α2-6-linked (FIG. 3A). The result is further confirmed by immunoprecipitation and lectin blotting using Sambucus nigra agglutinin (SNA), a lectin specific for α2-6-linked sialic acid (FIG. 3B).

To determine whether these sialic acids are carried on N- or O-linked glycans, Western blot analysis is performed using combined treatment with a STI and Peptide-N-Glycosidase F (PNGase F), an enzyme that cleaves all N-glycans. No additional band shift is observed upon combining PNGase F digestion with STI treatment (FIG. 3C), indicating that the sialic acids reside on N-glycans. This conclusion is supported by SNA pulldown experiments, where PNGase F treatment completely abolished the ability of SNA to precipitate IGF1R (FIG. 3D). Together, these results confirm that IGF1R carries α2-6-linked sialic acids exclusively on N-glycans.

Subsequent glycoproteomic analysis using high-resolution mass spectrometry identifies the specific glycosylation sites on IGF1R. Mass-spectral mapping reveals that asparagine residues 607 and 913 harbor sialylated N-glycans (Table 1 and FIG. 3G).

TABLE 1

| | | | Glycosylation | |
|---|---|---|---|---|
| Site | Subunit | Sialylated | frequency* | Glycan Component |
| N51CT | α | No | 70.00% | HexNAc₂Hex₇; HexNAc₂Hex₈; |
| | | | | HexNAc₂Hex₉ |
| N438LT | α | No | 95.83% | HexNAc₂Hex₇; HexNAc₂Hex₈; |
| | | | | HexNAc₂Hex₉; HexNAc₅Hex₅Fuc |
| N534VT | α | No | 100% | HexNAc₂Hex₆; HexNAc₂Hex₇; |
| | | | | HexNAc₂Hex₈; HexNAc₂Hex₉ |
| N607AS | α | Yes | 97.87% | HexNAc₅Hex₅; HexNAc₂Hex₆; |
| | | | | HexNAc₂Hex₈; HexNAc₂Hex₉; |
| | | | | HexNAc₃Hex₅NeuAc; HexNAc₃Hex₆; |
| | | | | HexNAc₄Hex₅NeuAc; HexNAc₄Hex₅Fuc; |
| | | | | HexNAc₅Hex₅; HexNAc₅Hex₅Fuc |
| N764IT | β | No | 53.85% | HexNAc₂Hex₈; HexNAc₂Hex₉ |
| N900YT | β | No | 100% | HexNAc₅Hex₅; HexNAc₂Hex₆; |
| | | | | HexNAc₂Hex₇; HexNAc₂Hex₈; |
| | | | | HexNAc₂Hex₉; HexNAc₃Hex₅Fuc; |
| | | | | HexNAc₃Hex₆ |
| N913GS | β | Yes | 100% | HexNAc₃Hex₅NeuAc; HexNAc₄Hex₅NeuAc |

*Glycosylation frequency = number of glycosylated peptide/number of total peptide; HexNAc, N-acetylhexosamine; Hex, hexose; Fuc, fucose; NeuAc, N-acetylneuraminic acid.

To assess whether IGF1R sialylation observed in ovarian cancer cells also occurs in other malignancies where ganitumab therapy has shown limited success, α2-6-linked sialylation of IGF1R is evaluated across multiple cancer cell lines. Notably, α2-6 sialylation of IGF1R is detected in lung, breast, and colorectal cancer cells (FIG. 3F). Furthermore, SNA pulldown assays demonstrate that STI treatment effectively inhibited α2-6 sialylation of IGF1R in cultured cells (FIG. 3E). These results collectively indicate that α2-6 sialylation of IGF1R is a conserved modification across several tumor types and that pharmacological inhibition of sialyltransferase activity can efficiently suppress this modification.

### Example 3. Asparagine 607 Sialylation of IGF1R Inhibits Binding and Response to Ganitumab

Among the two asparagine residues of IGF1R identified as sialylated (FIG. 3G), asparagine 607 is located in close proximity to the known Ganitumab-binding epitope. To investigate the role of asparagine 607 sialylation in IGF1R-Ganitumab interaction, an IGF1R mutant harboring a substitution of asparagine 607 to glutamine (IGF1R N607Q) is generated and expressed in IGF1R-knockout cells. Particularly, IGF1R has an amino acid sequence as stated in SEQ ID NO: 01, and IGF1R N607Q has an amino acid sequence as stated in SEQ ID NO: 02.

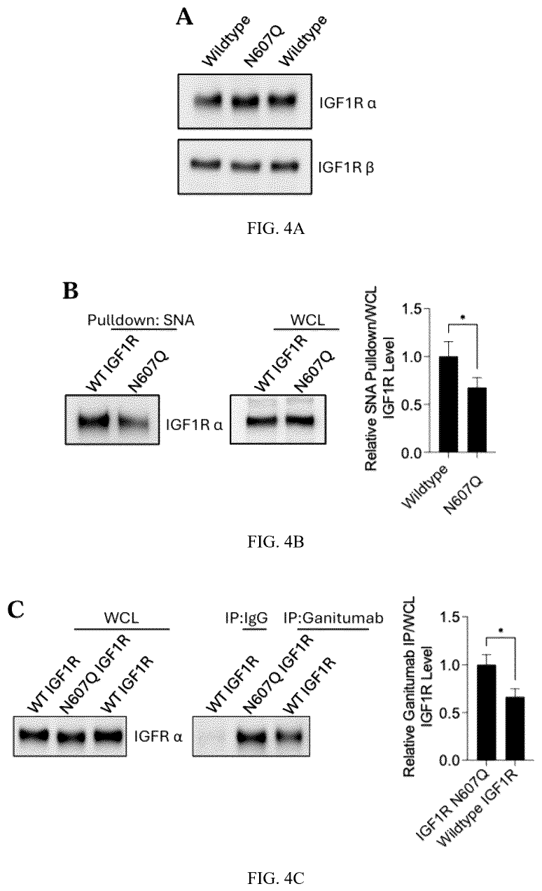

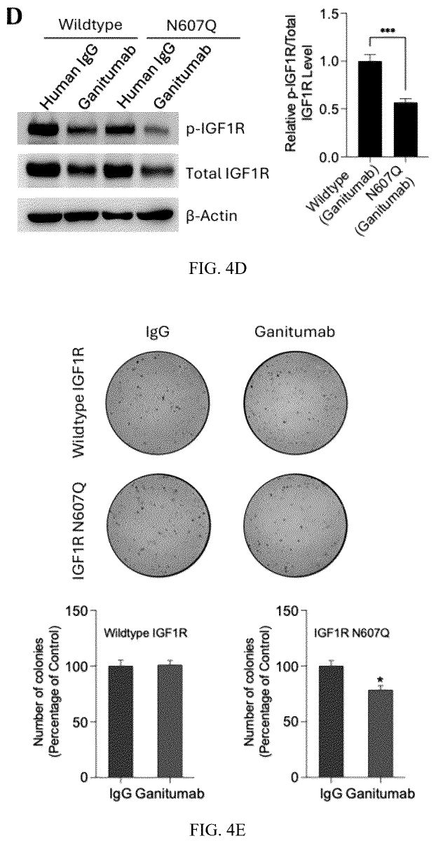

Compared to wild-type IGF1R, the IGF1R N607Q mutant exhibits a distinct band shift of the IGF1R α-subunit (FIG. 4A), indicating loss of N-glycosylation at this residue. Western blotting of SNA pulldown samples further confirms a marked reduction in α2-6 sialylation of IGF1R N607Q compared to wild-type IGF1R (FIG. 4B). Importantly, Ganitumab shows significantly enhanced immunoprecipitation efficiency and inhibitory activity toward the IGF1R N607Q mutant, as demonstrated by increased antibody binding and decreased receptor phosphorylation (FIG. 4C and FIG. 4D). These results indicate that α2-6 sialylation at asparagine 607 sterically or electrostatically impairs Ganitumab recognition and interferes with receptor deactivation. Furthermore, colony formation assays reveal that IGF1R N607Q mutant cells form markedly fewer colonies under Ganitumab treatment than wild-type controls (FIG. 4E), confirming that desialylation at residue 607 sensitizes ovarian cancer cells to Ganitumab.

### Example 4. Mutation of Asparagine 607 to Glutamine Sensitizes IGF1R to Ganitumab in an In Vivo Xenograft Model of Ovarian Cancer

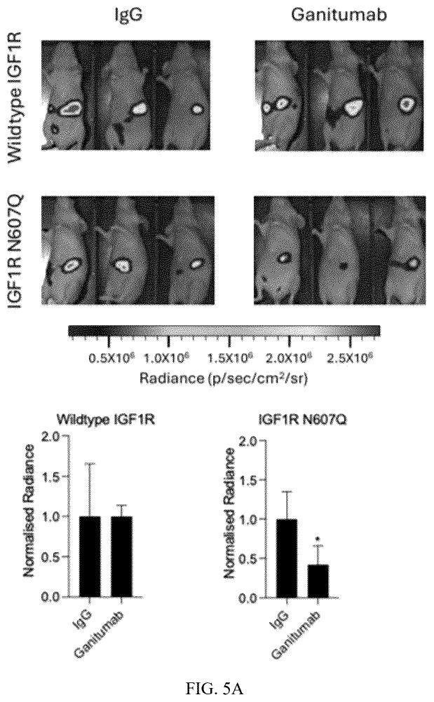

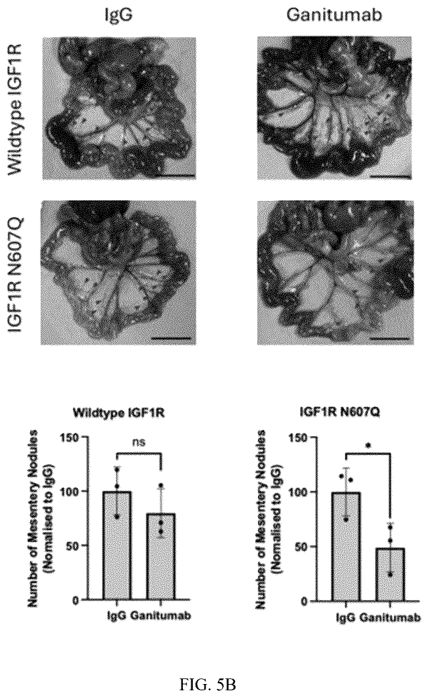

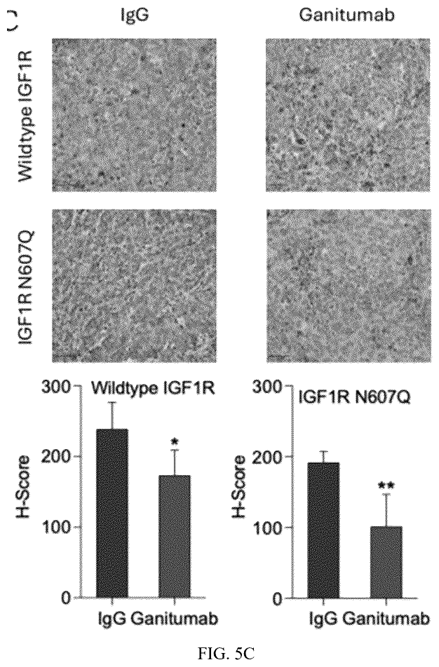

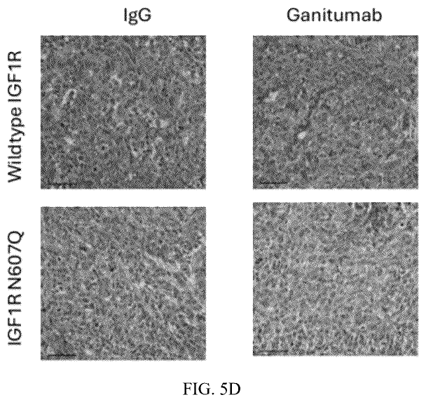

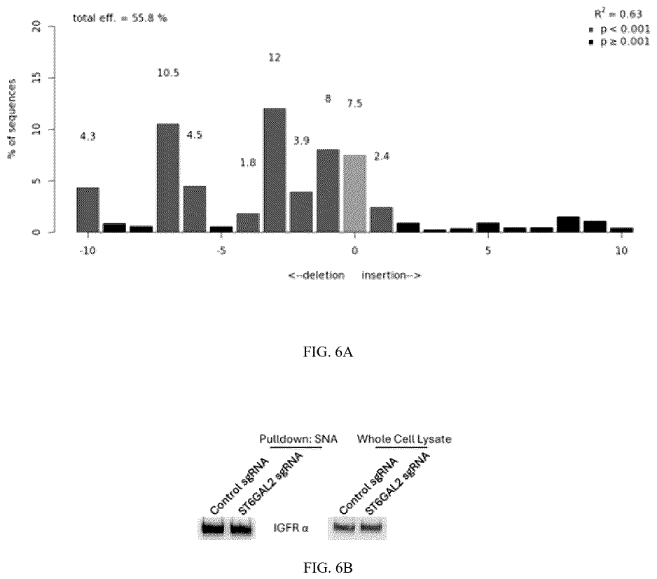

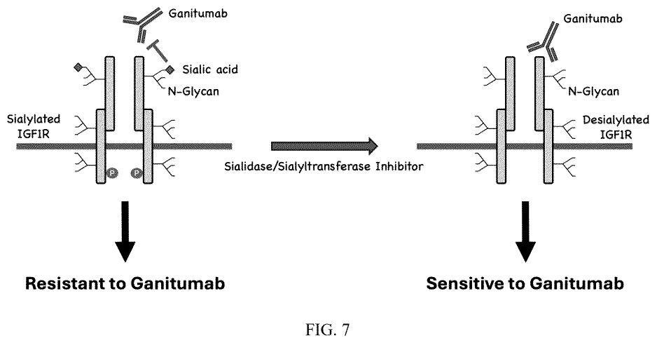

To determine whether desialylation of IGF1R at asparagine 607 confers therapeutic sensitivity in vivo, ovarian cancer cells harboring the IGF1R N607Q mutation are injected intraperitoneally into nude mice and treated with Ganitumab. Bioluminescence imaging demonstrate a significant reduction in tumor burden in mice bearing IGF1R N607Q-expressing tumors compared to those expressing wild-type IGF1R following Ganitumab administration (FIG. 5A). Consistent with this finding, the number of macroscopic tumor nodules on the mesenteric membrane is markedly lower in the N607Q group (FIG. 5B). Histological analysis of harvested omental tumors reveals a significant increase in IGF1R deactivation, as evidenced by reduced phospho-IGF1R immunoreactivity and lower H-scores in IGF1R N607Q tumors compared to wild-type (FIG. 5C). Collectively, these results demonstrate that loss of sialylation at asparagine 607 of IGF1R restores Ganitumab binding and receptor inhibition, thereby overcoming therapeutic resistance in ovarian cancer.

### Example 5. ST6GAL1-Mediated Sialylation of IGF1R Inhibits Ganitumab Binding in Ovarian Cancer Patients

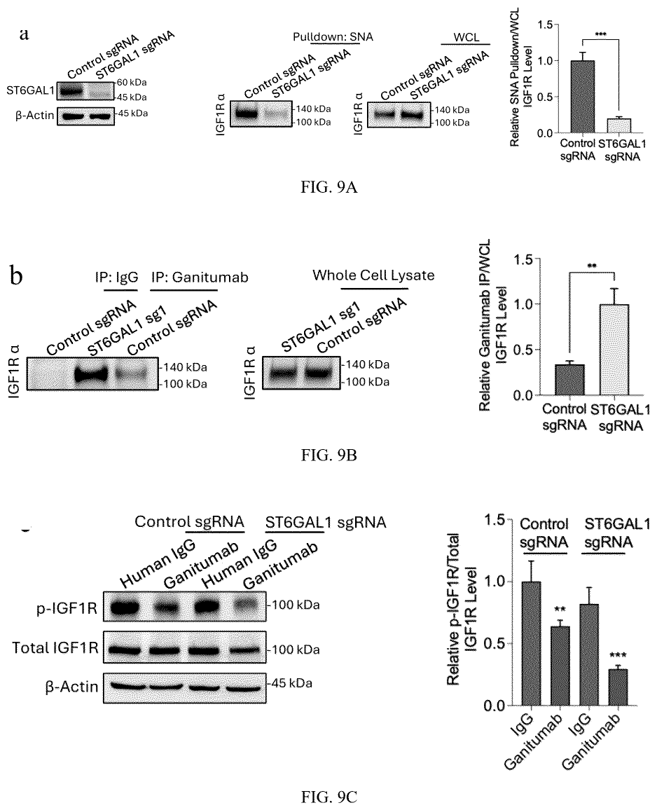

To elucidate the regulatory mechanism underlying IGF1R sialylation, the role of α2-6 sialyltransferases is investigated. In the human genome, two enzymes ST6GAL1 and ST6GAL2—are known to catalyze the addition of α2-6-linked sialic acids onto N-glycans. Using CRISPR/Cas9-mediated knockout combined with SNA pulldown assays, it is determined that ST6GAL1, but not ST6GAL2, is responsible for IGF1R sialylation (FIGS. 6A-6B and FIG. 9A). Loss of ST6GAL1 significantly enhances Ganitumab immunoprecipitation of IGF1R and increases receptor deactivation relative to control cells transduced with non-targeting sgRNA (FIG. 9B and FIG. 9C).

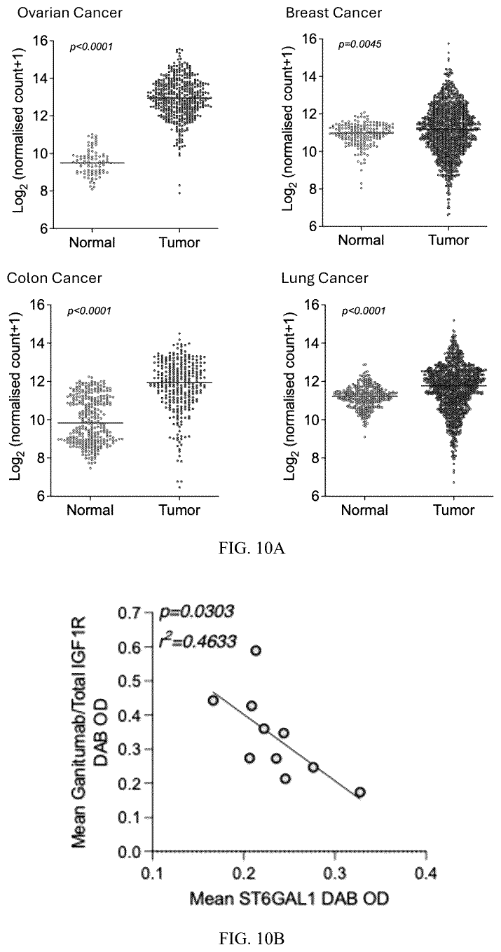

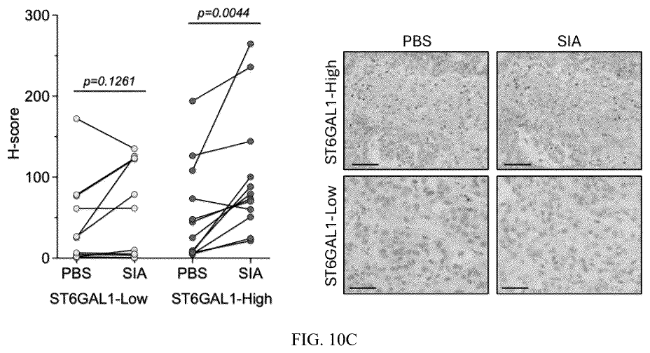

Gene expression profiling interactive analysis (GEPIA) of clinical datasets reveals that ST6GAL1 expression is more than threefold higher in ovarian cancer tissues compared to non-tumor tissues (FIG. 10A). In formalin-fixed paraffin-embedded (FFPE) sections of ovarian cancer patient tumors, sialidase digestion markedly increases Ganitumab staining intensity in samples with high ST6GAL1 expression (FIGS. 10B-10C), confirming that ST6GAL1-mediated α2-6 sialylation of IGF1R interferes with Ganitumab binding. These results establish ST6GAL1 as a key regulator of IGF1R glycosylation and as a determinant of therapeutic response to Ganitumab in ovarian cancer patients.

### Example 6. STI Prevents Addition of Sialic Acid and Improves Response to Ganitumab

SKOV-3 ovarian cancer cells are treated with 200 M 3Fax-Peracetyl-Neu5Ac (STI) for 24 hours, embedded in Histogel, and processed for paraffin embedding. Subsequent SNA staining (FIG. 8A) and SNA pulldown (FIG. 8B) analyses confirmed that STI treatment effectively inhibits the addition of sialic acid residues on IGF1R. Immunohistochemical staining with Ganitumab (FIG. 8C) further reveals that Ganitumab binds more strongly to STI-treated ovarian cancer cells than to untreated controls, whereas no appreciable change in binding is observed in normal ovarian surface epithelial (OSE) cells.

For in vitro efficacy assessment, SKOV-3 cells are pretreated with 200 M STI for 24 hours and subsequently subjected to a clonogenic survival assay under Ganitumab treatment. Co-treatment with STI and Ganitumab results in a pronounced reduction in colony formation and overall cell survival compared to either treatment alone, indicating that inhibition of sialylation sensitizes cancer cells to Ganitumab (FIG. 8D).

To further validate these results in vivo, 4- to 6-week-old nude mice are intraperitoneally injected with 3×10⁶ luciferase-expressing SKOV-3 cells and treated with either 6 mg/kg Ganitumab alone or in combination with 10 mg/kg STI. Bioluminescence imaging reveals significantly reduced tumor burden in the co-treated group compared to Ganitumab alone (FIG. 8E). Consistently, mesenteric tumor nodule counts show a marked decrease in tumor number following combination therapy (FIG. 8F). Together, these results demonstrate that STI effectively prevents IGF1R sialylation and synergistically enhances Ganitumab efficacy both in vitro and in vivo, providing a compelling rationale for combining sialyltransferase inhibition with anti-IGF1R antibody therapy in ovarian cancer.

### Example 7. ST6GAL1 is Upregulated in Multiple Cancers and is Responsible for Sialylation of IGF1R

CRISPR-Cas9 gene-editing technology is employed to knockout ST6GAL1 in SKOV-3 ovarian cancer cells. Transduction of Cas9-expressing SKOV-3 cells with sgRNA targeting ST6GAL1 results in successful knockout of ST6GAL1 (FIG. 9A). In one embodiment, the sgRNA has a sequence as stated in SEQ ID NO: 03. As shown in FIG. 9A, SNA pulldown analysis demonstrates that ST6GAL1 knockout effectively prevents the addition of sialic acid on IGF1R, confirming that ST6GAL1 is the principal sialyltransferase responsible for α2,6-linked sialylation of IGF1R.

To examine the impact of ST6GAL1 depletion on the interaction between IGF1R and Ganitumab, immunoprecipitation (IP) is performed using Ganitumab. As depicted in FIG. 9B, ST6GAL1-knockout cells exhibit markedly increased co-precipitation of IGF1R with Ganitumab compared to control cells, indicating enhanced antibody-receptor binding affinity in the absence of sialylation.

Functional analysis further reveals that ST6GAL1-knockout cells are more responsive to Ganitumab treatment. Western blot analysis of phosphorylated IGF1R (p-IGF1R) levels demonstrates a significant decrease in active IGF1R following Ganitumab treatment in ST6GAL1-deficient cells compared to wild-type controls, confirming improved receptor inhibition and therapeutic responsiveness (FIG. 9C).

### Example 8. ST6GAL1 Expression Correlates with Impaired Ganitumab Binding in Patient Samples

To investigate the clinical relevance of ST6GAL1-mediated sialylation, ST6GAL1 expression was analyzed in patient datasets and tumor sections. Using The Cancer Genome Atlas (TCGA) database, expression profiles of ST6GAL1 across multiple cancer types are evaluated. As shown in FIG. 10A, ST6GAL1 is significantly upregulated in several malignancies, including ovarian, breast, colorectal, and lung cancers, suggesting a broad role for ST6GAL1 in tumor pathophysiology and potential therapeutic resistance.

To determine whether ST6GAL1 expression influences Ganitumab binding in ovarian cancer, patient tumor samples are subjected to immunohistochemical staining with Ganitumab. Quantitative image analysis demonstrates an inverse correlation between ST6GAL1 expression and Ganitumab staining intensity (FIG. 10B). Tumors with high ST6GAL1 expression exhibit markedly weaker Ganitumab binding, indicating that elevated sialylation reduces antibody accessibility to IGF1R in patient tissues.

To further validate that this effect is mediated by sialylation, ST6GAL1-high ovarian tumor samples are treated with sialidase prior to Ganitumab staining. As shown in FIG. 10C, desialylation significantly enhances Ganitumab binding. These findings confirm that ST6GAL1-mediated α2,6-sialylation of IGF1R sterically hinders Ganitumab-IGF1R interaction in patient tumors. Collectively, the results establish ST6GAL1 expression as a biomarker for predicting Ganitumab responsiveness and highlight the therapeutic potential of targeting sialylation to restore anti-IGF1R antibody efficacy in cancer treatment.

The present invention employs a systematic series of molecular and biochemical experiments to precisely identify the key amino acid residue and glycoform responsible for ganitumab resistance. It is demonstrated that α2,6-sialylation at a specific asparagine residue of IGF1R is the critical determinant of reduced antibody-receptor interaction, and that enzymatic removal of this glycan effectively restores ganitumab binding and reverses therapeutic resistance. Importantly, the present invention further establishes that this glycomodification is specifically regulated by the sialyltransferase ST6Gal I and confirms, through analysis of patient-derived tumor samples, that elevated ST6GAL1 expression correlates with diminished ganitumab binding to tumor cells.

The present invention is the first to reveal a direct mechanistic link among ST6GAL1 expression, α2,6-sialylation, and the binding affinity of a clinically used monoclonal antibody within tumor cells. While previous studies have reported that N-glycans can hinder antibody recognition of immune checkpoint molecules such as PD-L1, these glycans represent a complex and heterogeneous group of modifications that are challenging to target selectively without affecting essential cellular functions. In contrast, sialylation represents a terminal and more accessible modification that can be specifically modulated. Accordingly, the present invention provides compelling evidence that future therapeutic strategies could benefit from selectively targeting sialylated IGF1R—for example, by employing a sialidase-conjugated form of ganitumab to locally remove sialic acids and enhance antibody-receptor binding.

Furthermore, the data disclosed herein offer critical insights into patient stratification. Tumors exhibiting high ST6GAL1 expression may be less responsive to ganitumab due to increased IGF1R sialylation, whereas patients with low ST6GAL1 expression are likely to exhibit enhanced therapeutic sensitivity. Thus, ST6GAL1 expression may serve as a predictive biomarker for selecting patients most likely to respond favorably to anti-IGF1R therapy, not only in ovarian cancer but also in other malignancies where hypersialylation contributes to therapeutic resistance.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

1. A method of treating a cancer in a subject in need thereof, comprising:
administering a pharmaceutically effective amount of a sialyltransferase inhibitor (STI) to the subject; and
administering a pharmaceutically effective amount of an insulin-like growth factor 1 receptor (IGF1R)-targeting agent to the subject;
wherein the STI inhibits the expression or enzymatic activity of ST6GAL1, thereby reducing α2,6-linked sialylation of the asparagine 607 residue of IGF1R, and enhancing the binding and inhibitory activity of the IGF1R-targeting agent.

2. The method of claim 1, wherein the STI comprises 3Fax-Peracetyl Neu5Ac or a pharmaceutically acceptable salt or derivative thereof or a small-molecule inhibitor of ST6GAL1.

3. The method of claim 1, wherein the IGF1R-targeting agent comprises Ganitumab.

4. The method of claim 1, wherein the IGF1R-targeting agent is an anti-IGF1R antibody, an IGF1R ligand-binding inhibitor, or a small molecule IGF1R tyrosine kinase inhibitor.

5. The method of claim 1, wherein the cancer comprises breast cancer, lung cancer, colorectal cancer, prostate cancer, pancreatic cancer, liver cancer, esophageal cancer, gastric cancer, ovarian cancer, cervical cancer, endometrial cancer, renal cancer, bladder cancer, thyroid cancer, melanoma, head and neck cancer, or sarcoma.

6. The method of claim 5, wherein the cancer is ovarian cancer.

7. The method of claim 1, wherein the subject exhibits elevated ST6GAL1 expression in the tumor tissue prior to treatment.

8. The method of claim 7, further comprising identifying a subject having a tumor with high ST6GAL1 expression or increased α2,6-sialylation of IGF1R, and selecting the subject for co-administration of the STI and IGF1R-targeting agent.

9. The method of claim 1, wherein the STI and the IGF1R-targeting agent are co-administered simultaneously, sequentially, or separately.

10. The method of claim 1, wherein the combined administration of the STI and the IGF1R-targeting agent produces a synergistic reduction in tumor growth relative to either treatment alone.

11. A pharmaceutical composition for treating cancer, comprising:
a pharmaceutically effective amount of a STI;
a pharmaceutically effective amount of an IGF1R-targeting agent; and
a pharmaceutically acceptable carrier or excipient.

12. The pharmaceutical composition of claim 11, wherein the STI comprises 3Fax-Peracetyl Neu5Ac or a pharmaceutically acceptable salt or derivative thereof or a small-molecule inhibitor of ST6GAL1.

13. The pharmaceutical composition of claim 11, wherein the IGF1R-targeting agent comprises Ganitumab.

14. The pharmaceutical composition of claim 11, wherein the IGF1R-targeting agent is an anti-IGF1R antibody, an IGF1R ligand-binding inhibitor, or a small molecule IGF1R tyrosine kinase inhibitor.

15. A kit of enhancing the therapeutic effect of an IGF1R-targeting agent, comprising:
a pharmaceutically effective amount of a STI; and
a pharmaceutically effective amount of an IGF1R-targeting agent;
wherein the STI inhibits the expression or activity of ST6GAL1, thereby preventing sialylation of IGF1R and improving binding of the IGF1R-targeting agent to IGF1R.

16. The kit of claim 15, wherein the STI comprises 3Fax-Peracetyl Neu5Ac or a pharmaceutically acceptable salt or derivative thereof or a small-molecule inhibitor of ST6GAL1.

17. The kit of claim 15, wherein the IGF1R-targeting agent comprises Ganitumab.

18. The kit of claim 15, wherein the IGF1R-targeting agent is an anti-IGF1R antibody, an IGF1R ligand-binding inhibitor, or a small molecule IGF1R tyrosine kinase inhibitor.


SEQUENCE LISTING (3 sequences)

SEQ ID NO: 1 - protein, 1367 aa, Homo sapiens
MKSGSGGGSPTSLWGLLFLSAALSLWPTSGEICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGDLCPGTMEEKPMCEKTTINNEYNYRCWTTNRCQKMCPSTCGKRACTENNECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCESDVLHFTSTTTSKNRIIITWHRYRPPDYRDLISFTVYYKEAPFKNVTEYDGQDACGSNSWNMVDVDLPPNKDVEPGILLHGLKPWTQYAVYVKAVTLTMVENDHIRGAKSEILYIRTNASVPSIPLDVLSASNSSSQLIVKWNPPSLPNGNLSYYIVRWQRQPQDGYLYRHNYCSKDKIPIRKYADGTIDIEEVTENPKTEVCGGEKGPCCACPKTEAEKQAEKEEAEYRKVFENFLHNSIFVPRPERKRRDVMQVANTTMSSRSRNTTAADTYNITDPEELETEYPFFESRVDNKERTVISNLRPFTLYRIDIHSCNHEAEKLGCSASNFVFARTMPAEGADDIPGPVTWEPRPENSIFLKWPEPENPNGLILMYEIKYGSQVEDQRECVSRQEYRKYGGAKLNRLNPGNYTARIQATSLSGNGSWTDPVFFYVQAKTGYENFIHLIIALPVAVLLIVGGLVIMLYVFHRKRNNSRLGNGVLYASVNPEYFSAADVYVPDEWEVAREKITMSRELGQGSFGMVYEGVAKGVVKDEPETRVAIKTVNEAASMRERIEFLNEASVMKEFNCHHVVRLLGVVSQGQPTLVIMELMTRGDLKSYLRSLRPEMENNPVLAPPSLSKMIQMAGEIADGMAYLNANKFVHRDLAARNCMVAEDFTVKIGDFGMTRDIYETDYYRKGGKGLLPVRWMSPESLKDGVFTTYSDVWSFGVVLWEIATLAEQPYQGLSNEQVLRFVMEGGLLDKPDNCPDMLFELMRMCWQYNPKMRPSFLEIISSIKEEMEPGFREVSFYYSEENKLPEPEELDLEPENMESVPLDPSASSSSLPLPDRHSGHKAENGPGPGVLVLRASFDERQPYAHMNGGRKNERALPLPQSSTC

SEQ ID NO: 2 - protein, 1367 aa, synthetic construct
  REGION 607: note = asparagine 607 of IGF1R is changed into glutamine 607
MKSGSGGGSPTSLWGLLFLSAALSLWPTSGEICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGDLCPGTMEEKPMCEKTTINNEYNYRCWTTNRCQKMCPSTCGKRACTENNECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCESDVLHFTSTTTSKNRIIITWHRYRPPDYRDLISFTVYYKEAPFKNVTEYDGQDACGSNSWNMVDVDLPPNKDVEPGILLHGLKPWTQYAVYVKAVTLTMVENDHIRGAKSEILYIRTQASVPSIPLDVLSASNSSSQLIVKWNPPSLPNGNLSYYIVRWQRQPQDGYLYRHNYCSKDKIPIRKYADGTIDIEEVTENPKTEVCGGEKGPCCACPKTEAEKQAEKEEAEYRKVFENFLHNSIFVPRPERKRRDVMQVANTTMSSRSRNTTAADTYNITDPEELETEYPFFESRVDNKERTVISNLRPFTLYRIDIHSCNHEAEKLGCSASNFVFARTMPAEGADDIPGPVTWEPRPENSIFLKWPEPENPNGLILMYEIKYGSQVEDQRECVSRQEYRKYGGAKLNRLNPGNYTARIQATSLSGNGSWTDPVFFYVQAKTGYENFIHLIIALPVAVLLIVGGLVIMLYVFHRKRNNSRLGNGVLYASVNPEYFSAADVYVPDEWEVAREKITMSRELGQGSFGMVYEGVAKGVVKDEPETRVAIKTVNEAASMRERIEFLNEASVMKEFNCHHVVRLLGVVSQGQPTLVIMELMTRGDLKSYLRSLRPEMENNPVLAPPSLSKMIQMAGEIADGMAYLNANKFVHRDLAARNCMVAEDFTVKIGDFGMTRDIYETDYYRKGGKGLLPVRWMSPESLKDGVFTTYSDVWSFGVVLWEIATLAEQPYQGLSNEQVLRFVMEGGLLDKPDNCPDMLFELMRMCWQYNPKMRPSFLEIISSIKEEMEPGFREVSFYYSEENKLPEPEELDLEPENMESVPLDPSASSSSLPLPDRHSGHKAENGPGPGVLVLRASFDERQPYAHMNGGRKNERALPLPQSSTC

SEQ ID NO: 3 - RNA, 20 nt, synthetic construct
  source 1..20: mol_type = other RNA
  misc_RNA 1..20: note = single guide RNA targeting ST6GAL1
tgtgaatgtatccatggtag